(19) United States
(12) Patent Application Publication
(10) Pub. No.: US 2013/0272956 A1
(43) Pub. Date: Oct. 17, 2013

(54) (11beta, 17alpha)-11-(4-(2-11C-ACETYL)PHENYL)-17,23-EPOXY-19,24-DINORCHOLA-4,9,20-TRIEN-3-ONE

(71) Applicants: Franciscus Theodorus Leonardus Brands, Oss (NL); Johannes Antonius Maria Hamersma, Oss (NL); Peter Hermanus Gerardus Wiegerinck, Oss (NL)

(72) Inventors: Franciscus Theodorus Leonardus Brands, Oss (NL); Johannes Antonius Maria Hamersma, Oss (NL); Peter Hermanus Gerardus Wiegerinck, Oss (NL)

(21) Appl. No.: 13/703,940

(22) PCT Filed: Jun. 17, 2011
(86) PCT No.: PCT/EP11/03012

§ 371 Date: Jun. 27, 2013

(30) Foreign Application Priority Data
Jun. 17, 2010 (EP) 10166364.9

(51) Int. Cl. C07J 53/00; A61K 51/04

(52) U.S. Cl. CPC C07J 53/001; A61K 51/0493
USPC 424/1.45; 540/28; 540/7

(57) ABSTRACT

The present invention relates to (11β,17α)-11-(4-(2-¹¹C-acetyl)phenyl)-17,23-epoxy-19,24-dinorchola-4,9,20-trien-3-one, an ¹¹C-labelled ORG 33628; a process for the preparation thereof, intermediates used in this process and the use of this compound as a PET tracer for the detection of breast cancer.

The present invention relates to (11β,17α)-11-(4-(2-¹¹C-acetyl)phenyl)-17,23-epoxy-19,24-dinorchola-4,9,20-trien-3-one, an ¹¹C-labelled ORG 33628; a process for the preparation thereof, intermediates used in this process and the use of this compound as a PET tracer for the detection of breast cancer.

### BACKGROUND

Breast cancer is a common cause of death among women and, although less common, also in men. Breast cancer is a well known example of a hormone dependent cancer in which steroid hormone receptors play a key role in tumor proliferation and disease progression. Steroid hormone receptors, more specifically estrogen receptors and progesterone receptors, are often expressed in breast tumor tissue.

ORG 33628 is a highly selective progesterone receptor modulator (PRM) with a predominant anti-progestagenic profile. Two main indications for the use of PRMs are the treatment of breast cancer and fertility regulation. The effect of Org 33628 was tested in relevant models for these indications and the compound has been considered to be an interesting option for the prevention and treatment of breast cancer and for fertility control (Kloosterboer et al., 2000, Steroids 65 pp 733-740). ORG 33628 is suggested to be useful in the treatment of hormone responsive breast cancer (WO2009/134723).

Currently, early detection of breast cancer, which is crucial to improve disease-free survival, occurs via mammography. Mammography is the process of using low-dose amplitude-X-rays to examine the human breast and is used as a diagnostic as well as a screening tool. The goal of mammography is the early detection of breast cancer, typically through detection of characteristic masses and/or micro calcifications. Early detection is believed to reduce mortality from breast cancer.

Imaging-based tumor detection such as positron emission tomography (PET) has been proposed as an alternative method for the early detection of breast cancer (Hospers et al., 2008, Current Pharmaceutical Design, 14, pp 3020-3032). PET is a non-invasive nuclear imaging technique that allows monitoring and quantification of hormone receptor expression across lesions throughout the body. PET detection of breast cancer requires radio-active tracers that specifically bind to breast cancer tissue before decay of the compound due to the half-life of the radio isotope.

Early detection methods for breast cancer based on radio-labeled ORG 33628 as a PET tracer will allow for an earlier detection of breast cancer than mammography.

### SUMMARY OF THE INVENTION

The present invention provides a compound according to Formula 1, i.e. (11β,17α)-11-(4-(2-¹¹C-acetyl)phenyl)-17,23-epoxy-19,24-dinorchola-4,9,20-trien-3-one.

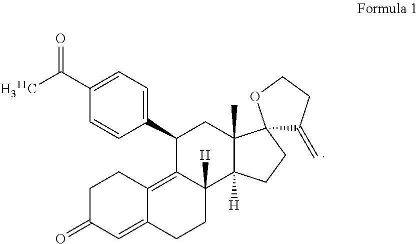

### BRIEF DESCRIPTION OF THE DRAWINGS

FIG. 1 is the analytical HPLC chromatogram showing the fraction containing [¹¹C] of a compound according to Formula 1, r.t. 29.32.

### DETAILS OF THE INVENTION

The present invention provides a compound according to Formula 1, i.e. (11β,17α)-1′-(4-(2-¹¹C-acetyl)phenyl)-17,23-epoxy-19,24-dinorchola-4,9,20-trien-3-one.

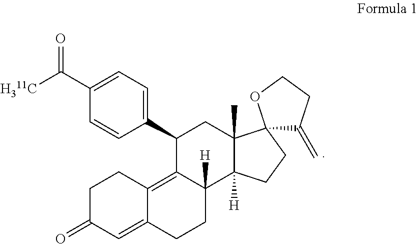

A further embodiment of the invention relates to the process for preparation of a compound of Formula 1, comprising the steps a through e according to Scheme

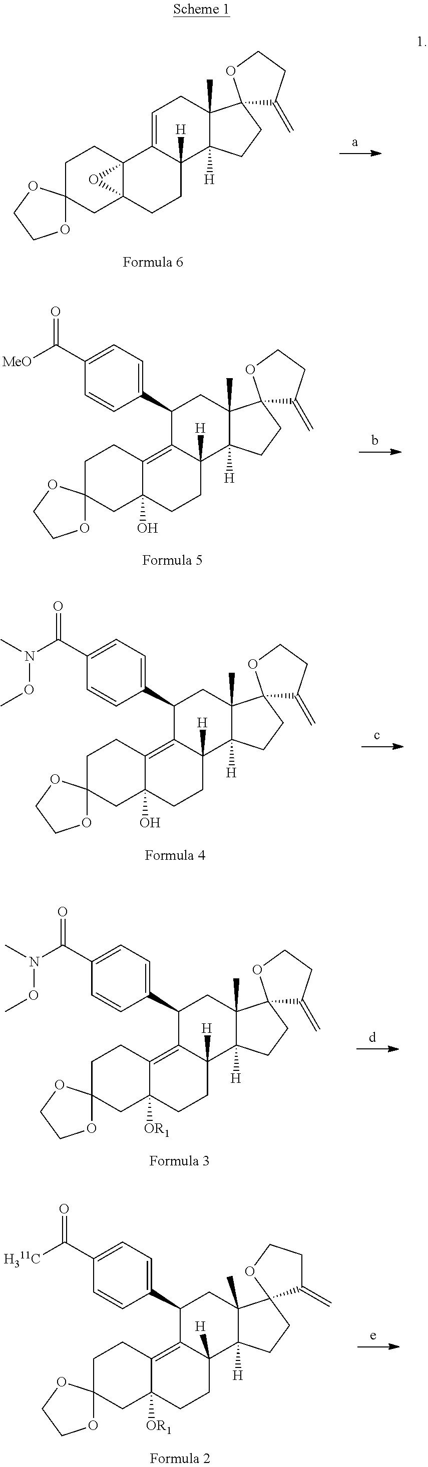

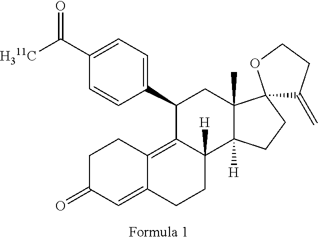

wherein
R1 is an acid labile hydroxyl-protecting group;
in step a. a compound of Formula 5 is obtained by a Copper-mediated reaction of the compound of Formula 6 with the reaction product of methyl 4-halogen-benzoate and a Grignard reagent;
in step b. the compound of Formula 5 is reacted with N—O-dimethylhydroxylamine and a base to obtain a product of Formula 4;
in step c. the compound of Formula 3 is prepared from a compound of Formula 4 wherein the hydroxyl group in the compound of Formula 4 is protected with an acid labile hydroxyl-protecting group R1;
in step d. the compound of Formula 3 is reacted with [11C]methyl lithium to obtain a compound according to Formula 2;
in step e. the compound of Formula 2 is treated with an acid to obtain a compound of Formula 1.

Acid labile hydroxyl-protecting groups are known to the person skilled in the art. Preferably, acid labile hydroxyl-protecting groups are selected from a group consisting of tetrahydropyrinyl-group, ethoxyethyl-group, tri(C1-4)alkyl silyl group, wherein each (C1-4)alkyl group is independently selected from methyl, ethyl, n-propyl, iso-propyl, n-butyl, iso-butyl or tert-butyl.

In one aspect the halogen in methyl 4-halogen-benzoate is a bromine or iodine, iodine being preferred.

The compound of Formula 6 is known for example from Jiang et al, 2006, Bioorganic & Medicinal Chemistry, 14, pp 6726-6732.

In particular, the invention relates to the process described above in Scheme 1 wherein the acid treatment in step e involves the treatment of the compound of Formula 2 with an aqueous acidic solution. More particularly, the aqueous acidic solution may be added to the reaction mixture of the previous reaction step after the reaction step d has run to completion. Even more particularly, the aqueous acidic solution in step e. is selected from a group consisting of aqueous HCl solution and aqueous H₂SO₄ solution.

In another aspect the invention relates to a process for the preparation of a compound according to Formula 3 comprising the steps of a to c according to Scheme 2

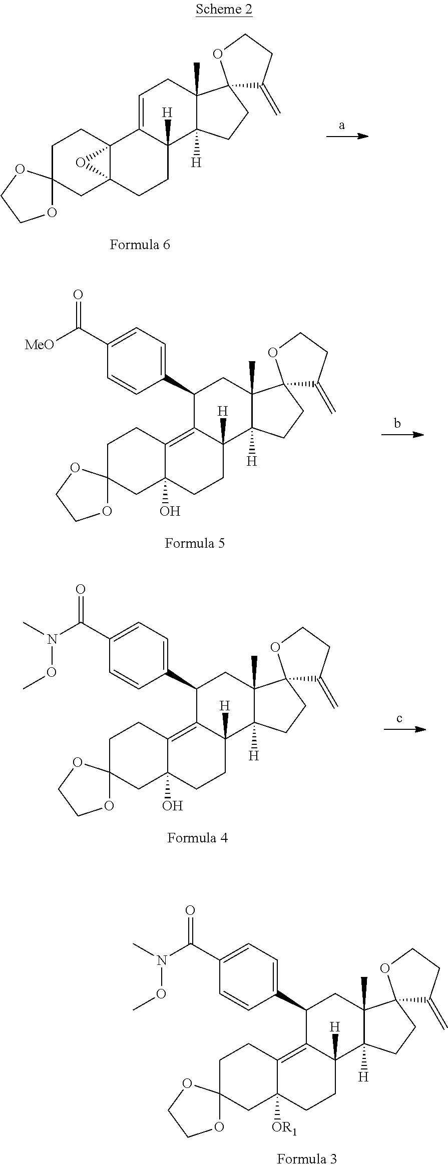

wherein
R1 is an acid labile hydroxyl-protecting group defined as in Scheme 1;
in step a. a compound of Formula 5 is obtained by a Copper-mediated reaction of the compound of Formula 6 with the reaction product of methyl 4-halogen benzoate and a Grignard reagent;
in step b. the compound of Formula 5 is reacted with N,O-dimethylhydroxylamine and a base to obtain a product of Formula 4;
in step c. the compound of Formula 3 is prepared from a compound of Formula 4 wherein the hydroxyl group in the compound of Formula 4 is protected with an acid labile hydroxyl-protecting group R1.

In one aspect the halogen in methyl 4-halogen-benzoate is a bromine or iodine, iodine being preferred.

In another aspect the Copper mediated reaction in step a. of Scheme 1 or 2 involves the presence of one or more Cu²⁺ salts. More particularly, step a. involves the presence of Cu(OAc)₂.

In another aspect, the invention relates to the process described above in Scheme 1 or 2, wherein in step c the acid labile hydroxyl-protecting group is a tri(C1-4)alkyl silyl group, more preferably, said protecting group is a trimethyl silyl group.

In another aspect the invention relates to step b. in the process described above in Scheme 1 or 2 wherein the base is an organometallic nucleophile. In yet another aspect the base is a Grignard reagent.

In another aspect, the invention relates to process described above in Scheme 1 or 2 wherein the Grignard reagent in step a. or the base in step b. is isopropylmagnesium bromide.

The synthesis of ORG 33628—which is an unlabeled compound according to Formula 1—and formulations comprising this compound are known (EP277676, EP549041 or EP582338). The synthesis route provided in these references is not suitable for the preparation of the ¹¹C-labelled compound of the invention due to the short half-life of the ¹¹C isotope.

In yet another aspect, the invention relates to a compound according to Formula 2 wherein R1 is an acid labile hydroxyl-protecting group as defined in Scheme 1.

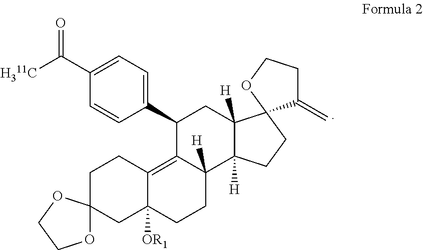

In yet another aspect the invention relates to a compound according to Formula 3.

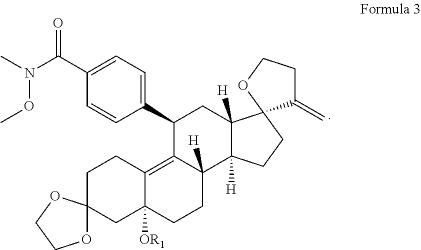

In another aspect, the invention relates to a compound according to Formula 4.

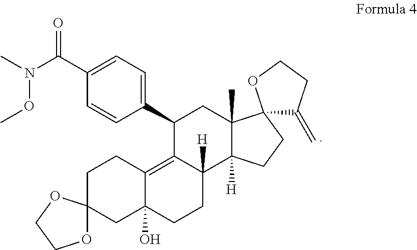

In yet another aspect, the invention relates to a compound according to Formula 5.

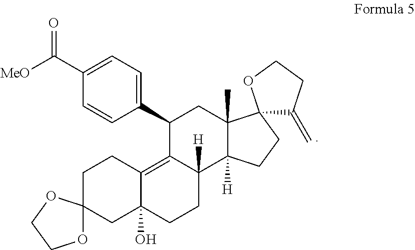

A further embodiment of the invention is a process for the preparation of a compound according to Formula 1 comprising the steps of
a. reacting a compound according to Formula 3 wherein R1 is an acid labile hydroxyl-protecting group as defined in Scheme 1.

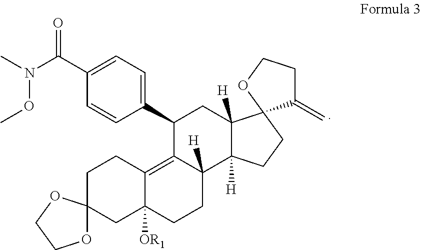

with [¹¹C]methyl lithium to obtain a compound according to Formula 2 wherein R1 is an acid labile hydroxyl-protecting group as defined in Scheme 1; and

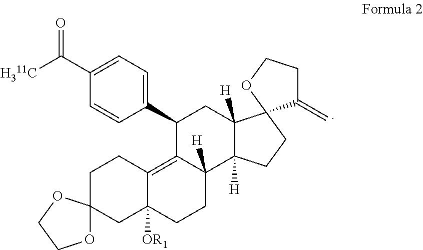

b. comprising the additional step of an acid treatment of compound according to Formula 2 wherein R1 is an acid labile hydroxyl-protecting group as defined in Scheme 1 to obtain a compound according to Formula 1.

In particular, the invention relates to the process described above wherein the acid treatment involves the treatment of the compound of Formula 2 with an aqueous acidic solution.

More particularly, the invention relates to the processes described above wherein a compound of Formula 3 is converted in a compound of Formula 1, wherein the acid may be added to the reaction mixture of the previous reaction step. Said addition should be done during work-up of the reaction product obtained after the reaction step with [¹¹C]Methyl Lithium. More particularly, said aqueous acidic solution in these processes is selected from a group consisting of aqueous HCl solution and aqueous H₂SO₄ solution.

In another aspect the invention relates to the processes described above wherein the [¹¹C]methyl lithium is prepared by reacting [¹¹C]methyl halide with alkyl-lithium. Preferably, the halide is bromide or iodide, iodide being more preferred; and said alkyl is a (C3-6)alkyl group.

A (C3-6)alkyl group is defined as a branched or unbranched alkyl group having 3-6 carbon atoms, being propyl, isopropyl, butyl, isobutyl, sec-butyl, tert-butyl, pentyl, isopentyl, sec-pentyl, tert-pentyl, hexyl, iso-hexyl, sec-hexyl and tert-hexyl. (C3-4)alkyl groups being preferred. (C4)alkyl groups being more preferred, n-butyl being most preferred.

In another aspect, the invention relates to the processes described above wherein the [¹¹C]methyllithium is prepared by reacting [¹¹C]methyl iodide with n-butyllithium.

As used in this document, the term halogen means fluorine, chlorine, bromine or iodine. The term halide means fluoride, chloride, bromide or iodide.

In a further embodiment the invention relates to a method of diagnosing or monitoring breast cancer or a breast cancer state in mammals comprising the steps of:
a. administering an effective amount of the compound according to Formula 1 to a subject;
b. imaging said mammal by performing a PET scan on the subject to generate PET scan images and diagnosing or monitoring said breast cancer or breast cancer state.

In another aspect, the invention relates to a method for detecting breast cancer in mammals comprising the step of performing a PET scan to generate PET scan images on a subject to whom an effective amount of the compound of Formula 1 was pre-delivered.

In yet another aspect, the invention relates to a compound according to Formula 1 for detecting breast cancer. In particular the invention relates to a compound according to Formula 1 for pre-operative or post-operative detection of breast cancer.

In another embodiment, the invention relates to the use of the compound according to Formula 1 for detecting breast cancer. In particular the invention relates to the use of a compound according to Formula 1 for pre-operative or post-operative detection of breast cancer.

The compound according to Formula 1 can be used in therapy.

A further aspect of the invention resides in the use of the compound according to Formula 1 for the manufacture of a medicament to be used for the radio-labeling of cancer tissue, in particular of breast cancer tissue.

A further aspect of the invention resides in the use of the compound according to Formula 1, for the manufacture of a medicament to be used for detecting cancer, in particular breast cancer.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen is bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

The compounds of this invention include the hydrates or solvates of the compounds listed.

The present invention also relates to a pharmaceutical composition comprising the compound according to Formula 1 in admixture with pharmaceutically acceptable auxiliaries and optionally other therapeutic agents. The auxiliaries must be “acceptable” in the sense of being compatible with the other ingredients of the composition and not deleterious to the recipients thereof.

The invention further includes a compound of formula 1 in combination with one or more other drug(s).

Compositions include e.g. those suitable for subcutaneous, intravenous, intramuscular, or local administration, and the like, all in unit dosage forms for administration.

Mixed with such pharmaceutically acceptable auxiliaries, e.g. as described in the standard reference, Gennaro, A. R. et al., Remington: The Science and Practice of Pharmacy (20th Edition., Lippincott Williams & Wilkins, 2000, see especially Part 5: Pharmaceutical Manufacturing), the active agent may be applied, by means of pharmaceutically acceptable liquids, as a fluid composition, e.g. as an injection preparation, in the form of a solution, suspension or emulsion.

For parenteral administration, aqueous suspensions, isotonic saline solutions and sterile injectable solutions may be used, containing pharmaceutically acceptable dispersing agents and/or wetting agents, such as propylene glycol or butylene glycol. In addition, for parenteral administration, the pharmaceutical composition of the invention may be presented in unit-dose or multi-dose containers, e.g. injection liquids in predetermined amounts, for example in sealed vials and ampoules.

The exact dose and regimen of administration of the active ingredient, or a pharmaceutical composition thereof, may vary with the particular compound, the route of administration, and the age and condition of the individual subject to whom the medicament is to be administered.

In general parenteral administration requires lower dosages than other methods of administration which are more dependent upon absorption. The dosage of administration may differ between a female and a male recipient.

¹¹C containing molecules have a short half-life. Hence there is a need to provide a compound of Formula 3 in a formulation that can be converted in a compound of Formula 1 just before it is used as a PET tracer. The quantity of the compound according to Formula 3 in such a formulation should be sufficient to convert it into the radio-labeled compound of this invention, and administer said radio-labeled compound to a person in need thereof, and to perform a PET scan on said person.

The invention further includes a stable formulation of a compound according to Formula 3. In another aspect, the invention relates to a stable formulation of a compound according to Formula 3 in combination with packaging material suitable for said formulation. Stability for such a formulation is defined as the compound of Formula 3 being stable long enough to allow for normal storage and transportation periods.

In the compounds of the invention, except for the ¹¹C-labeled carbon atom, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples.

The invention also relates to those compounds or processes wherein all specific definitions for alkyl, R1, halide, and halogen in the various aspects of the is inventions defined here above occur in any combination within the definition Scheme 1 or 2 or the compounds of Formula 2 or 3.

The invention is illustrated by the following examples which are given by way of representation and not limitation.

### EXAMPLES

Example names refer to reaction steps in Scheme 1 wherein R1 is an acid labile hydroxyl-protecting group as defined before.

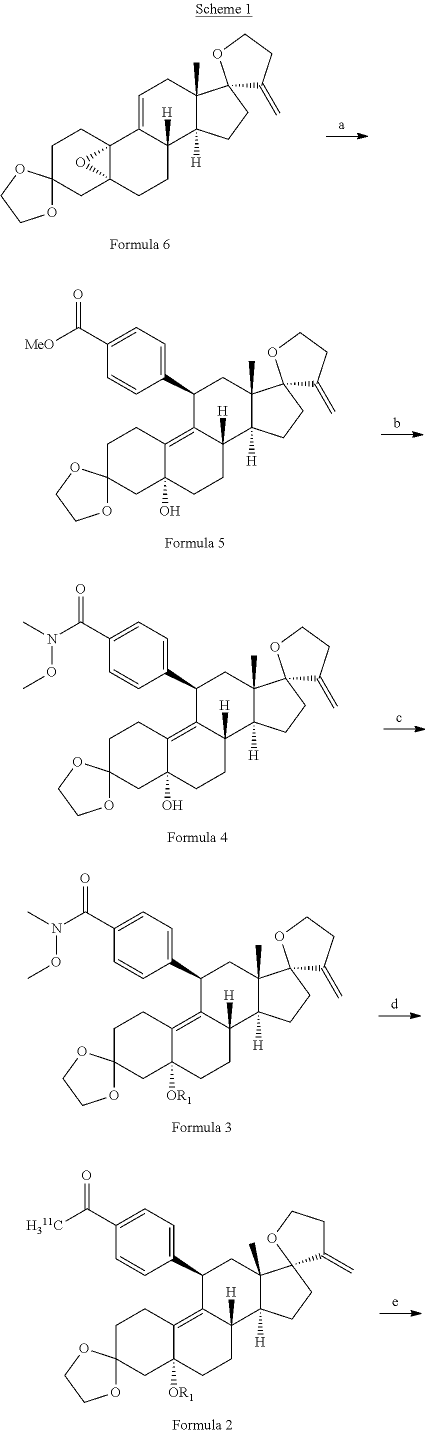

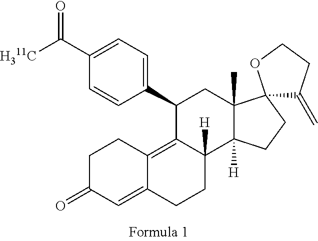

### Example 1

### Step a

5.1 g. Methyl 4-iodobenzoate was dissolved in 50 ml THF and cooled to −30° C. Isopropylmagnesium bromide (11.0 ml, 1.6 M in THF) was added. After addition of the isopropylmagnesium bromide the color of the solution changed from slight yellow to clear yellow. The conversion was followed on GC-MS. This freshly prepared Grignard solution was added to a solution of 2.5 g. of a compound of Formula 6 and 118 mg Cu(OAc)₂ in 40 ml THF at 10° C. while keeping the Grignard solution at −30° C. during the addition. The solution turned from blue (a compound of Formula 6 and Cu(OAc)₂ in THF) to green during addition. After the addition the solution was colored brown/black. After 10 minutes the reaction was completed. The reaction mixture was quenched with 100 ml of an aqueous NH₄Cl solution (10%). The crude product was extracted twice with toluene and the organic layers were washed with 100 ml of an aqueous NaCl solution (20%). The organic layers were dried with MgSO₄ and concentrated to a final volume of 25 ml. The crude solution was purified by column chromatography (80 g. silica: heptane/EtOAc+1% pyridine) with a gradient from heptane/EtOAc 9:1 to 1:1 in 40 minutes, followed by heptane/EtOAc 1:1 for 20 minutes. Relevant fractions were combined and concentrated. A compound of Formula 5 (2.7 g) was obtained as white foam. ¹H-NMR, CDCl₃, δ0.50 (s, 3H), δ1.15 (m, 1H), δ1.30 (m, 1H), δ1.40 (m, 1H), δ1.60 (m, 2H), δ1.70 (m, 2H), δ1.90 (m, 1H), δ2.05 (m, 4H), δ2.19 (m, 2H), δ2.30 (dd, 1H), δ2.65 (m, 3H), δ3.80 (m, 2H), δ3.90 (s, 3H), δ3.92 (m, 2H), δ4.00 (m, 2H), δ4.22 (d, 1H), δ4.40 (s, 1H), δ4.82 (d, 1H), δ5.10 (d, 1H), δ7.28 (d, 2H), δ7.93 (d, 2H).

### Example 2

### Step b

Dimethylhydroxylamine (618 mg) and 2.2 g. of a compound of Formula 5 was suspended in 22 ml of THF and cooled to −20° C. Isopropylmagnesium bromide (11.3 ml, 1.5 M in THF) was added dropwise while stirring at −20° C. After 15 minutes stirring the reaction was finished. The reaction mixture was quenched with 40 ml of an aqueous solution of NH₄Cl (10%) and extracted twice with EtOAc. The combined organic layers were washed with water and an aqueous solution of NaCl (20%), dried over MgSO₄ and concentrated under reduced pressure. A white foam remained, crude compound of Formula 4 in a yield of 2.2 g.

The crude product was purified with column chromatography, 40 g. of silica, heptane/EtOAc+1% pyridine with a gradient from heptane/EtOAc 9:1 to heptane/EtOAc 1:1 (30 minutes) followed by heptane/EtOAc 1:1 (30 minutes). Relevant fractions were combined and concentrated. A white solid was characterized as a compound of Formula 4, (1.8 g). ¹H-NMR, CDCl₃, δ0.51 (s, 3H), δ1.20 (m, 1H), δ1.40 (m, 1H), δ1.60 (m, 3H), δ1.70 (m, 2H), δ1.86 (m, 1H), δ2.00 (m, 4H), δ2.20 (m, 2H), δ2.33 (m, 1H), δ2.64 (m, 3H), δ3.35 (s, 3H), δ3.52 (s, 3H), δ3.80 (m, 2H), δ3.92 (m, 2H), δ4.00 (m, 2H), δ4.22 (d, 1H), δ4.39 (s, 1H), δ4.82 (t, 1H), δ5.11 (t, 1H), δ7.22 (d, 2H), δ7.59 (d, 2H).

### Example 3

### Step c

A compound of Formula 4 (1.8 g) was dissolved in 20 ml of pyridine and cooled to 0° C. while stirring. Chlorotrimethylsilane (8.19 mmol, 1.039 ml, 0.889 g) was added and the reaction mixture was allowed to warm up to room temperature and stirred for an additional 30 minutes. Water was added to the reaction mixture and the mixture was extracted twice with toluene. The combined organic layers were washed with water and with an aqueous NaCl solution (20%), dried over MgSO₄ and concentrated under reduced pressure, yielding a compound of Formula 3 (R1 being trimethyl silyl) as white crystals (1.9 g). ¹H-NMR, CDCl₃, δ0.00 (s, 9H), δ0.32 (s, 3H), δ0.97 (m, 1H), δ1.20 (m, 1H), δ1.40 (m, 3H), δ1.50 (m, 2H), δ1.70 (m, 1H), δ1.80 (m, 4H), δ1.90 (m, 2H), δ2.00 (m, 2H), δ2.49 (m, 1H), δ3.19 (s, 3H), δ3.38 (s, 3H), δ3.65 (m, 2H), δ3.74 (m, 2H), δ4.05 (d, 1H), δ4.69 (t, 1H), δ4.96 (t, 1H), δ7.05 (d, 2H), δ7.41 (d, 2H).

### Example 4

### Step d and e

[¹¹C]Carbon dioxide was produced by the ¹⁴N(p,α)¹¹C nuclear reaction using an IBA 18/9 cyclotron. The target gas used was nitrogen containing 0.5% oxygen. [¹¹C]Methyl iodide was synthesized from [¹¹C]carbon dioxide. The formed [¹¹C]methyl iodide was transferred via sodium hydroxide and phosphorus pentoxide columns to the reaction vial (septum equipped, 3 mL) and bubbled through a solution with a compound of Formula 3 (R1 being trimethyl silyl) (2.3 mg, 4.2 pmol) in THF (anhydrous, 250 μL) at −50° C. Once the transfer was complete the vial was cooled down to −70° C. and n-butyllithium (10 μL, 1.6 M) was added with a syringe to convert the [¹¹C]methyl iodide to [¹¹C]methyllithium. After 1 min reaction time the vial was heated to 20° C. The solution was allowed to react for 1 min before HCl(aq) (100 μL, 1.0 M) was added with a syringe; after 1 min the reaction was completed. The crude reaction mixture was diluted with MeOH/water 50/50 (1.5 mL) prior to purification by preparative HPLC. Preparative HPLC was performed on a Nucleosil C18 column 250×16 mm, mobile phase 80/20 MeOH/H2O, 7 ml/min, UV detection 254 nm. Analytical HPLC was performed on a Platinum C18 column 4,6×250 mm, mobile phase 80/20 MeOH/H2O, 1 ml/min, UV detection 254 nm.

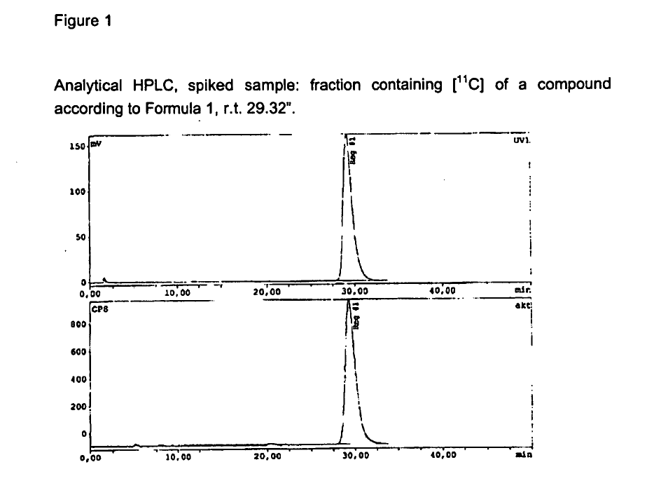

A compound according to Formula 1, yield 8% decay-corrected radio chemical yield was isolated and was characterized using HPLC. Unlabeled ORG 33628 was used as reference compound. In FIG. 1 the analytical HPLC chromatogram is shown “Analytical HPLC, spiked sample: fraction containing [¹¹C] of a compound according to Formula 1, r.t. 29.32”.

The activity in the reaction vial was measured in a dose calibrator after i) the transfer of [¹¹C]methyl iodide, ii) purging of the vial with helium subsequent to the alkylation reaction and iii) completion of the deprotection step. Analytical samples were taken after the alkylation reaction and at the end of the deprotection.

### Example 5

### Unlabeled Compound According to Formula 2 (R1 Being Trimethyl Silyl)

In an analogues manner as example 4 step d, a compound of Formula 2 (R1 being trimethyl silyl) was obtained when using methyl lithium and without the addition of HCl(aq). In this way an unlabeled compound of Formula 2 (R1 being trimethyl silyl) was obtained. ¹H-NMR, CDCl₃, δ0.00 (s, 9H), δ0.32 (s, 3H), δ0.94 (m, 1H), δ1.20 (m, 1H), δ1.40 (m, 3H), δ1.50 (m, 2H), δ1.70 (m, 1H), δ1.84 (m, 4H), δ1.90 (m, 2H), δ2.00 (m, 2H), δ2.40 (s, 3H), δ2.49 (m, 1H), δ3.65 (m, 2H), δ3.74 (m, 2H), δ4.05 (d, 1H), δ4.69 (t, 1H), δ4.96 (t, 1H), δ7.10 (d, 2H), δ7.69 (d, 2H).

1. (canceled)

2. A process for preparing a compound of Formula 1, comprising the steps (a) through (e) according to Scheme 1 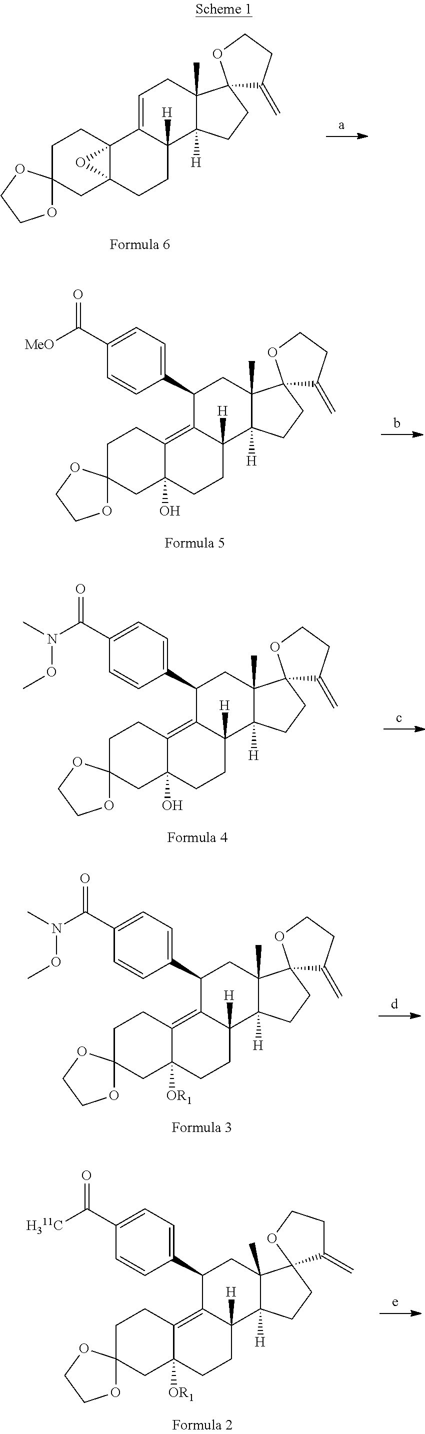 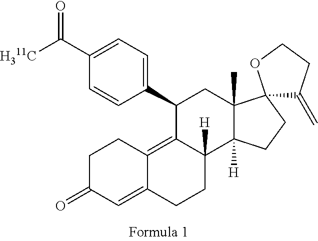
wherein
R1 is an acid labile hydroxyl-protecting group;
in step a. a compound of Formula 5 is obtained by a Copper-mediated reaction of the compound of Formula 6 with the reaction product of methyl 4-halogen-benzoate and a Grignard reagent;
in step b. the compound of Formula 5 is reacted with N,O-dimethylhydroxylamine and a base to obtain a product of Formula 4;
in step c. the compound of Formula 3 is prepared from a compound of Formula 4 wherein the hydroxyl group in the compound of Formula 4 is protected with an acid labile hydroxyl-protecting group R1;
in step d. the compound of Formula 3 is reacted with [11C]Methyl lithium to obtain a compound according to Formula 2; and
in step e. the compound of Formula 2 is treated with an acid to obtain a compound of Formula 1.

3. A compound selected from:
a compound according to Formula 1 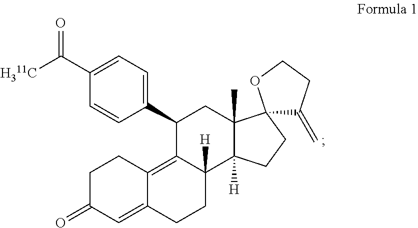
a compound according to Formula 2, wherein R1 is an acid labile hydroxyl-protecting group 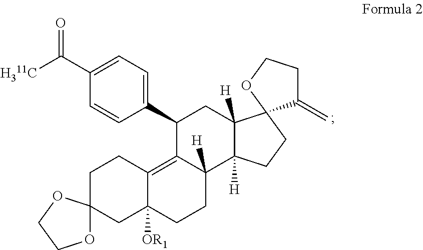
a compound according to Formula 3 wherein, R1 is an acid labile hydroxyl-protecting group 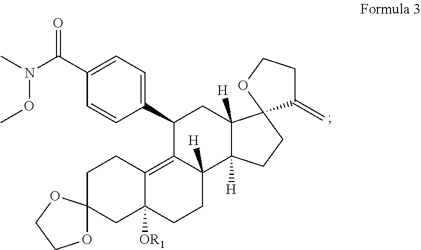
a compound according to Formula 4 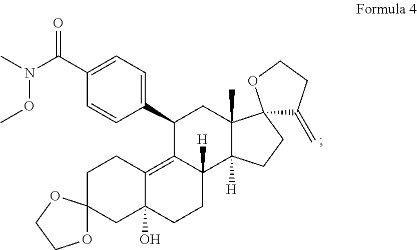
and,
a compound according to Formula 5

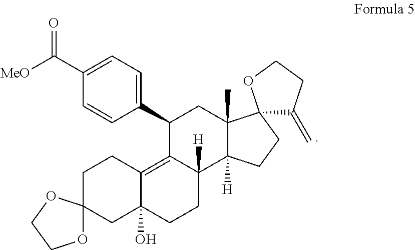

4-8. (canceled)

9. The process according to claim 2 wherein the acid treatment is combined with the previous reaction step.

10. The process according to claim 2 wherein in step b the base is a Grignard reagent.

11. The process according to claim 10 wherein the Grignard reagent is isopropylmagnesium bromide.

12. The process according to claim 2 wherein the [¹¹C]methyllithium is prepared by reacting [¹¹C]methyl halide with (C3-6)alkyl-lithium.

13. A method of diagnosing or monitoring breast cancer or a breast cancer state in mammals comprising the steps of:
a. administering an effective amount of the compound according to Formula 1 to a subject;
b. imaging said mammal, performing a PET scan on the subject to generate PET scan images and diagnosing or monitoring said breast cancer or breast cancer state.

14-15. (canceled)

16. The compound according to claim 3 which is [chemical structure]
